(19) United States
(12) Patent Application Publication
(10) Pub. No.: US 2019/0046704 A1
(43) Pub. Date: Feb. 14, 2019

(54) BLOOD PUMP, PREFERABLY FOR SUPPORTING A HEART

(71) Applicant: Berlin Heart GmbH, Berlin (DE)

(72) Inventors: Leonid Choub, Berlin (DE); Kurt Graichen, Berlin (DE); J?rg Müller, Berlin (DE); Peter Nüsser, Kleinmachnow (DE); Adrian Wisniewski, Berlin (DE)

(73) Assignee: Berlin Heart GmbH, Berlin (DE)

(21) Appl. No.: 15/758,789

(22) PCT Filed: Sep. 12, 2016
(86) PCT No.: PCT/EP2016/071404

§ 371 Date: Mar. 9, 2018

(30) Foreign Application Priority Data
Sep. 11, 2015 (EP) 15184856.1

(51) Int. Cl. A61M 1/10; A61M 1/12; F04D 13/02

(52) U.S. Cl. CPC A61M 1/1015; F04D 13/024; A61M 1/122; A61M 1/1036

(57) ABSTRACT

A blood pump is provided that comprises a housing with an inlet arranged upstream, an outlet arranged downstream, and a rotatably mounted rotor with an axis and a blading. The rotor comprises a cylinder bushing or a cylinder, wherein the blading is arranged on an outer surface of the cylinder. The rotor is magnetically mounted in an axial direction and comprises at least a first ring, which is secured to the blading, runs radially externally around the blading, and is magnetised in the axial direction, and also a second magnetised ring portion, which runs externally around the first ring, for forming an axial magnetic bearing.

The present application relates to a blood pump according to the preamble of claim 1.

Blood pumps, for example for assisting a heart, are used in patients with cardiac insufficiency or vascular weakness. The blood pumps described in this application can be used for example as ventricular assist devices (VADs) in order to assist the left ventricle, the right ventricle, or for both ventricles in the case of a system having two pumps.

Various blood pumps are known in the prior art. For example, US 2014/0322020 describes a VAD having a radial pump. Radial pumps have a rotor with a blading, wherein the blading widens radially from a pump inlet to a pump outlet.

Axial pumps are sufficiently known in the prior art. For example, document U.S. Pat. No. 8,668,473 B2 presents a pump having a hydrodynamically mounted rotor, wherein permanent magnets are incorporated into the blading of the rotor as appropriate. An axial mounting of the axial pump is made possible due to the combination of hydrodynamic and magnetic bearing.

The object of the present application is to provide a blood pump of simple structure.

The object is achieved in accordance with the invention by a blood pump for example according to claim 1.

In one embodiment, the blood pump comprises a housing with an inlet arranged upstream, an outlet arranged downstream, and a rotor, which is mounted rotatably between the inlet and the outlet and which has an axis of rotation and a blading. The rotor is magnetically mounted in the axial direction.

The rotor also comprises a cylinder bushing or a cylinder or hub, wherein the blading is arranged on an outer surface of the cylinder bushing or the cylinder. A cylinder bushing differs from a cylinder in that it has a cylindrical or truncated cone-shaped recess as considered from the axis. In some variants the rotor can have the shape of a cylinder from its end arranged upstream to its end arranged downstream. In other exemplary embodiments the cylinder or the cylinder bushing can reach from the end of the blading arranged upstream to the end of the blading arranged downstream. In some exemplary embodiments the rotor can taper conically towards its end arranged downstream. A cylinder or a cylinder bushing has, along a portion, a height, which runs coaxially with the rotor axis, of at least one inner or outer wall which delimits the cylinder or the cylinder bushing, wherein the radial distance of the inner and/or outer wall from the rotor axis is substantially constant. In particular, it is provided in some exemplary embodiments that the blading is arranged at least partially on the cylindrical or cylinder bushing-like portion.

The rotor also comprises a first ring, which is secured to the blading, runs radially externally around the blading, and is magnetised in the axial direction. Correspondingly hereto, a second magnetised ring portion running externally around the first ring is provided, which second ring portion is housed in a first axial housing portion of the housing. The first magnetised ring, which is secured to the blading, forms a magnetic axial bearing in conjunction with the second magnetised ring portion.

Here as well, it is provided in some exemplary embodiments that the first ring is arranged at least partially in the cylindrical or cylinder bushing-like portion. In some exemplary embodiments the first ring is arranged parallel to the outer wall of the rotor. In some exemplary embodiments it is also provided that the first ring is present in the form of a cylinder bushing. The distance of an inner wall of the first ring and an outer wall of the rotor is also substantially constant.

In other exemplary embodiments it is provided that the blading is present between the outer wall of the rotor and the first ring. Here, the first ring preferably does not extend over the entire axial extent of the blading, but only over an axial portion of the blading. Here, as considered upstream, the first ring can extend flush with the blading, and the rotor can extend axially after the downstream side of the rotor.

In further exemplary embodiments the first ring runs parallel to an inner wall of the inlet, which in the region of the rotor can describe an inlet of uniform diameter.

Furthermore, the rotor comprises further permanent magnets, which are used to set the rotor in rotation by means of the stator. These permanent magnets are not identical to the magnetised portions of the ring. The magnetised portions are not used for drive, but instead as a bearing.

The use of a first ring, which runs radially externally around the blading, in conjunction with the second ring portion oriented correspondingly thereto offers a particularly good coupling on account of the symmetry of the ring and the small axial distance between the first ring and the second ring portion, and therefore offers an axial mounting that can be produced particularly easily. In some exemplary embodiments the axial mounting of the rotor in the housing in addition to the magnetic mounting does not comprise any further bearings, such as a hydrodynamic bearing. In other variants, in addition to the magnetic axial bearing, a further bearing, for example a hydrodynamic bearing, can also be provided.

In some embodiments the first ring or the second ring portion for example can consist of or comprise soft iron or permanent magnets. Here, in some embodiments at least one of the two elements of the first ring and of the second ring portion is constructed of a permanent magnet in order to achieve improved coupling between the first ring and the second magnetised ring portion.

In some embodiments the first ring is arranged at the upstream end of the rotor. Here, the ring can be flush or approximately flush in the axial direction with the start, arranged upstream, of the cylindrical portion of the rotor. In a further embodiment the second magnetised ring portion can likewise be formed as a closed ring. In this case, an arrangement of the first and second ring relative to one another can be produced particularly easily. The axial magnetisation of the first ring should deviate from the magnetisation direction of the further ring by 180°. A passive axial magnetic mounting is formed as a result.

In a further embodiment the length of the first ring in the axial direction is smaller than the length of the second ring portion in the axial direction.

In a further embodiment the first housing portion, in which the upstream part of the rotor is arranged, is formed substantially as a cylinder bushing. In this way, the pump can be mounted in a particularly simple way, in particular in the embodiments in which there is no further inlet guide vane or outlet guide vane provided. The rotor can be “inserted” substantially easily into the inlet and is fixed in the desired position.

In a further embodiment the blood pump comprises a further rotor portion, which is provided with a third ring portion, preferably a third ring. Correspondingly to the third ring portion, a fourth ring portion corresponding to the third ring portion is arranged in the housing in the second axial housing portion. With the presence of a further ring pair, the effect of the axial bearing can be intensified. In a further embodiment the first and the third ring portion are distanced axially from one another. Here, the first ring portion can be arranged at an upstream end of the blading and the third ring portion can be arranged at a downstream end of the blading.

In a further embodiment the blading is configured in the form of at least one spiral. In the present case, a spiral is understood to mean a blading running helically around the rotor outer surface. Here, it is advantageous in some embodiments when the blading comprises two or more spirals, which, as considered in the circumferential direction, are arranged at regular distances from one another on the outer surface of the cylinder bushing or the cylinder.

In some exemplary embodiments it is also provided that a spiral in each case extends along the rotor at least with an encircling angle of 360°.

In a further embodiment the thickness of the blading is smaller than the width of the flow channel which extends between two spirals and through which the fluid is transported. The distance between two spirals is measured here transversely to the axis of the rotor. This distance is wider here than the thickness of one of the spirals.

In a further embodiment the rotor is mounted in the axial direction exclusively by the axial magnetic bearing. Here, the term “exclusively” is to be understood to mean that motor-related bearing effects are also included by the axial magnetic bearing. Here, the axial magnetic bearing can be formed as a passive axial magnetic bearing, i.e. without active control of the position or as active axial magnetic bearing. In the case of an active axial magnetic bearing a control coil is arranged in the housing for example upstream and/or downstream of the second ring portion, with which control coil the magnetic force acting on the magnetised first ring can be adjusted, and the position of the rotor therefore likewise can be adjusted. In other embodiments, another bearing, for example a hydrodynamic bearing or a mechanical bearing or a bearing combined of these bearings, can also be provided alternatively or additionally to the magnetic bearing.

In a further embodiment the axial magnetic bearing is formed in such a way that a downstream end of the blading is mounted fully upstream of the flow outlet. This embodiment is possible both in the case of pure axial pumps, which convey the fluid in an outlet arranged coaxially with the inlet, and in the case of what is known as a tangential pump, the outlet of which is rotated by an angle of more than 45°, preferably between 80° and 100°, relative to the inlet. Here, the part of the pump adjoining the cylindrical portion in which the rotor of the blood pump is mounted is referred to as the outlet, provided the blood can be pushed into the outlet merely by the additional radial component. In one embodiment the rotor is adjoined by a spiral outlet, which in particular receives the circumferential component of the speed and recovers static pressure by deceleration of the speed.

In a further embodiment the blading is arranged on the rotor in such a way that a downstream end of the rotor lies downstream of a downstream end of the blading. In other words, the rotor extends downstream further than the blading, i.e. a downstream portion of the rotor is free from blading. This can have the advantage, inter alia, that the flow conditions can be stabilised.

In a further embodiment a radial distance between an inner surface of the housing and an outer surface of the first ring portion is such that a hydrodynamic bearing is formed between the inner surface of the housing and the outer surface of the first ring portion. This hydrodynamic bearing acts in the radial direction and stabilises the rotor radially. Here, a bearing is understood to mean that a hydrodynamic bearing can perform the radial bearing alone. In other variants the hydrodynamic bearing can be formed in such a way that it is provided additionally to a further radial bearing, for example a magnetic radial bearing.

In a further embodiment a radial distance between an inner surface of the housing and an outer surface of the first ring is selected in such a way that there is no hydrodynamic bearing formed between the inner surface of the housing and the outer surface of the first ring. This can be the case for example when the distance between said outer surface and inner surface is great. Nevertheless, the ring in this case can act as a radial damping member in that impacts acting on the rotor which lead to a vibration of the rotor are damped hydrodynamically by the ring and the movement of the rotor is stabilised. Whether the bearing is now exclusively supporting or is merely a damping member is thus dependent on the distance between the inner surface of the housing and the outer surface of the first ring and the axial dimension of the ring pair.

In some exemplary embodiments a mandrel is arranged between the inlet and the outlet, which mandrel extends preferably upstream from the outlet. The rotor also has a cylinder bushing and is mounted rotatably on the mandrel. Examples of a blood pump of this kind can be found for example in the application having the same filing date and the internal reference 157EP 0367. Here, the mandrel is often formed as a mandrel extending in a cylindrical manner into the housing. At its upstream end, the mandrel can be rounded in order to form a lower flow resistance for the fluid to be conveyed. In the embodiments which provide a mandrel on which the rotor is rotatably mounted, a hydrodynamic bearing can be formed in some variants between an outer surface of the mandrel and an inner surface of the rotor. This hydrodynamic bearing preferably performs the radial mounting of the rotor within the blood pump. In the embodiments which provide a mandrel, a stator driving the rotor can be arranged in the mandrel. Furthermore, both in the exemplary embodiments with and without mandrel, the stator can be arranged in the first housing portion in a manner running radially externally around the rotor. In some exemplary embodiments it is also provided that the stator is arranged proximally, i.e. downstream, of the rotor.

In a further embodiment the mandrel and the housing or the interior defined by the housing are oriented coaxially with one another at least in part.

Further exemplary embodiments and variants will be explained with reference to the following drawings and description of the drawings, in which:

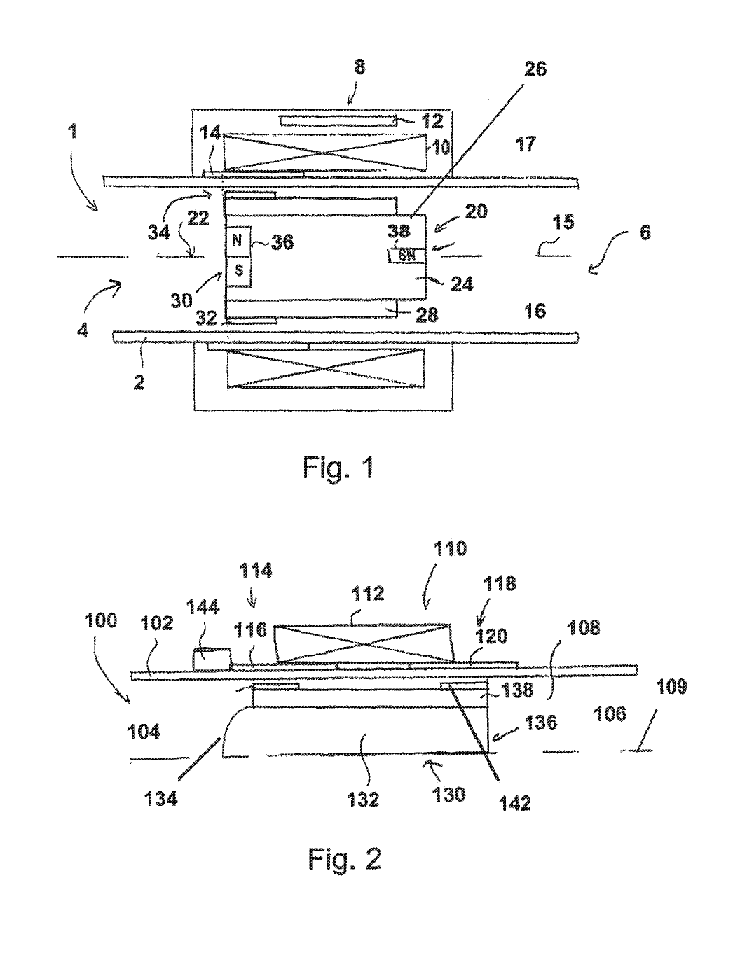

FIG. 1 shows a longitudinal section through an embodiment of a pump;

FIG. 2 shows a further embodiment of a pump in longitudinal section;

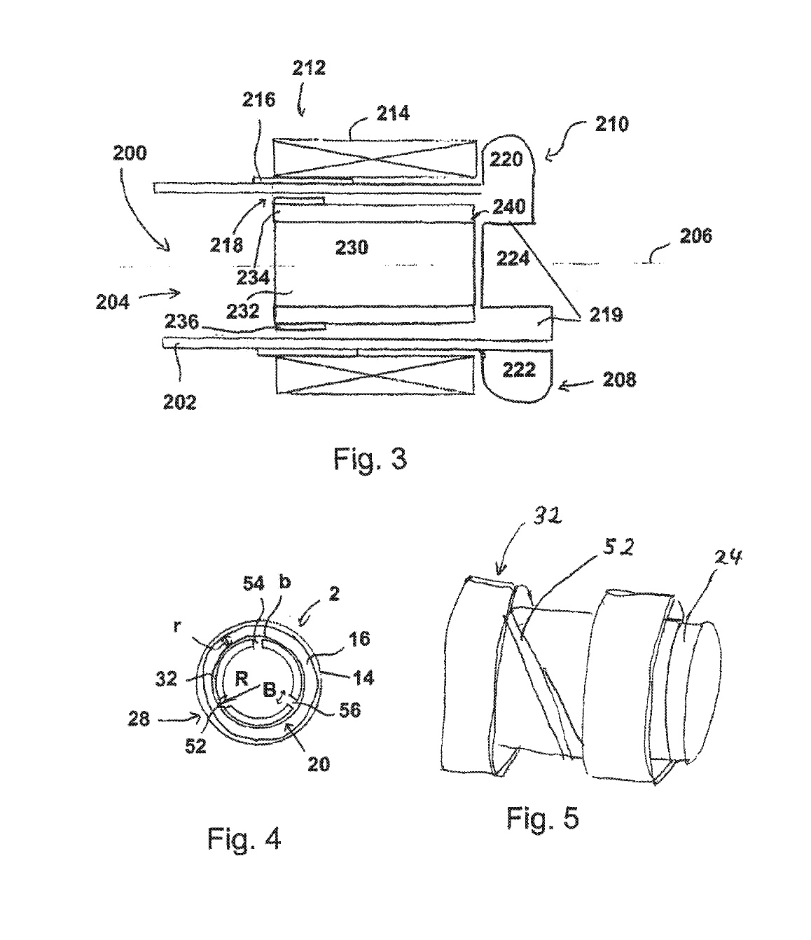

FIG. 3 shows a further embodiment of a pump in longitudinal section;

FIG. 4 shows a cross-section through a rotor as can be used in the pumps of FIG. 1 or 2;

FIG. 5 shows a further embodiment of a rotor for the pumps;

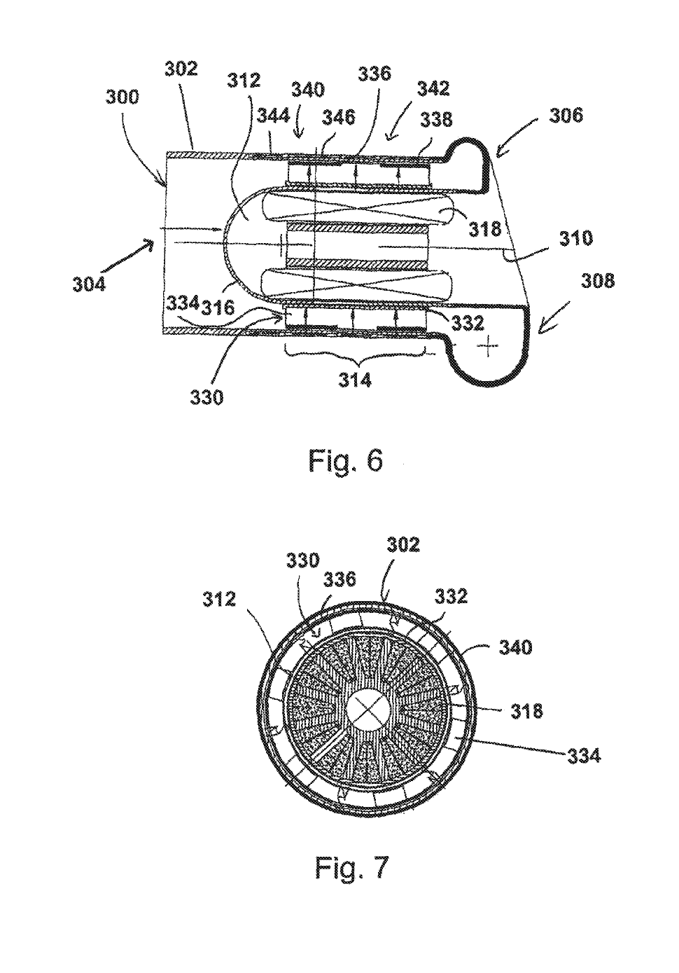

FIG. 6 shows a longitudinal section through a pump with a rotor mounted on a mandrel;

FIG. 7 shows a cross-section through the pump of FIG. 6.

FIG. 1 shows a longitudinal section through an embodiment of a pump. The blood pump 1 comprises a housing 2, which has an inlet 4 arranged upstream, and an outlet 6 arranged downstream. A stator 10 is disposed in a first axial housing portion 8 and is configured to set a rotor, which will be explained in greater detail hereinafter, in rotation. The first axial housing portion 8 also comprises a pump controller 12, which is designed to control the stator, for example. The illustrated blood pump 1 can be connected via the pump controller 12 to further components, such as a battery, a charging device or a control unit, by means of a cable. In the first axial housing portion 8 there is additionally a magnetised ring portion 14, which is magnetised in the axial direction. Here, the magnetic north pole is upstream and the magnetic south pole is downstream. The housing 2 is designed rotationally symmetrically about the axis 15. This is true both for the magnetised ring portion 14 formed as a ring and for the stator 10. The pump controller 12 merely spans a portion within the axial housing portion 8. A rotor 20 is arranged in an interior 16 of the housing 2, and the axis 22 of said rotor runs coaxially to the axis 15 of the housing 2. The rotor 20 comprises a cylinder 24, with a blading 28 arranged on the outer surface 26 thereof. The blading 28 extends from the upstream end 30 of the rotor 20 downstream, wherein, however, the blading ends upstream of the downstream end of the rotor. However, the blading can also start downstream of the start of the rotor and can extend downstream along the hub. The blading 28 is formed in the present example by a plurality of spirals, for example three spirals. A first ring 32 is arranged at the upstream end 30 of the rotor or of the portion of the blading 28 arranged in this region. This ring is made for example of soft iron. In conjunction with the magnetised ring portion 14, the ring 32 forms an axial magnetic bearing 34. The ring 32 could alternatively also be formed as a permanent magnet. Here, it is provided in one embodiment to select the magnetisation against the direction of magnetisation of the magnetised ring portion 14. Further components are arranged within the cylinder 24 of the rotor 20. For example, permanent magnets 36 which can set the rotor in rotation by means of the stator 10 can be arranged in the interior of the cylinder 24. These permanent magnets are oriented here in a north-to-south or south-to-north direction as considered transversely to the axis 22. Furthermore, further permanent magnets 38 can be arranged within the cylinder 24 and can be used substantially as part of a sensor system. The further permanent magnets 38 for example can be used, in conjunction with a sensor coil not shown in FIG. 1, to detect changes in position of the rotor in the axial direction and/or to bring about such changes by means of at least one control coil (not shown) and to forward these to the pump controller.

In the present exemplary embodiment both the interior 16 of the housing 2 and the rotor 20 are substantially cylindrical. There is no need for any tapering of the interior 16 due to the selected passive axial mounting.

The rotor 20 can be made for example of titanium or other biocompatible materials. Titanium or another biocompatible material likewise lends itself as a material for the housing. The axial housing portion 8 is fluidically tight with respect to the surrounding environment 17 outside the pump. Although an axial pump with inlet and outlet oriented coaxially with one another is shown in FIG. 1, the rotor, the stator and the axial magnetic bearing can be used in conjunction with an inlet and an outlet rotated relative to the inlet, as will be described later for example in FIG. 5.

An arrangement of the rotor within the interior of the housing 2 alternative to FIG. 1 is shown in FIG. 2. The blood pump 100 comprises a housing 102 with an inlet 104 and an outlet 106. The inlet 104 and the outlet 106 are oriented coaxially with one another. The interior 108 of the housing 102 is formed rotationally symmetrically as a cylinder and is illustrated extending merely radially outwardly from the axis 109. A stator 112 is arranged in a housing portion 110 and basically performs the functions of the stator 10 of FIG. 1. Furthermore, the housing portion 110 comprises a magnetised ring 106 in its upstream portion 114 and a further ring 120 in its downstream portion 118, the magnetisation of said further ring being opposite the magnetisation of the ring 116. The rotor 130 comprises a cylindrical portion 132 and an upstream and downstream end 134 and 136 respectively, which are shaped substantially in the form of a segment of a sphere. In some embodiments this improves the flow from the middle of the rotor 130 to the radially outer blading 138. A ring 140, which is arranged correspondingly to the ring 116, is disposed at the upstream end of the blading 138. The rotor 130 also comprises a further ring 142, which corresponds with the ring 120. Both the ring 140 and the ring 142 are formed by magnets, in particular permanent magnets, and their magnetisations are opposite the magnetisations of the rings corresponding to them. Alternatively, the rings can comprise soft iron or other magnetisable materials.

In a further embodiment the rings 116 and 120 are divided into two substantially in the axial direction and comprise two rings magnetised oppositely to one another. This arrangement of the rings also causes a passive magnetic axial bearing.

In the blood pump 100 of FIG. 2 a sensor coil 144 is additionally arranged, which can derive a position of the rotor on the basis of a voltage induced by an axial movement of the rotor. In another embodiment the coil 144 is a control coil, which, by means of active energisation with current, generates a magnetic field which for example interacts with the rings 140 and 142 and thus permits an active positioning of the rotor in the interior. In a further embodiment both a sensor coil and a control coil are provided. In addition, two sensor coils or also two control coils can be provided.

A further blood pump 200 is illustrated in FIG. 3. The blood pump 200 is constructed similarly to the blood pump 1. However, the blood pump 200 has a housing 202, which, besides an inlet 204, which runs along an axis 206, has an outlet 208, which is rotated relative to the inlet by 90°. The output 208 comprises a spiral chamber 210, as is illustrated for example in FIGS. 3a, 3b, 7a, 7b, 7c and 8 of application U.S. Pat. No. 2,014,017 172. The details shown and described there are incorporated fully into the disclosure of this application. The spiral chamber 210 adjoins the axial housing portion 212, in which the stator 214 and the magnetised ring 216 of the axial bearing 218 are disposed. Here, the spiral chamber has a radial widening, and runs in a spiralled manner from the portion 219 to the portion 220 to the outlet 222. A hub stump 224 extends upstream from the spiral chamber. The rotor 230 comprises a cylinder 232 and a blading 234 and a ring 236, as has already been described in conjunction with FIGS. 1 and 2. The blading 234 extends from the upstream end 238 to the downstream end 240 of the rotor 230. Here, the rotor 230 is arranged in the axial housing portion 212 in such a way that the hub stump 224 forms a stop. Here, the rotor is arranged relative to the hub stump in such a way that it does not protrude into the spiral chamber 210. For example, a rotor, as described in conjunction with FIG. 1 or with FIG. 2, can therefore also be used in the blood pump 200. The hub stump 224 can extend over the entire axial length of the spiral chamber 210. In further exemplary embodiments the rotor protrudes into the spiral chamber, wherein the downstream end of the blading, however, does not protrude into the spiral chamber. The outlet 208 is then disposed between the upstream end and the downstream end of the rotor 230.

A cross-section through the pump 1 or 100 or 200 in the region of the axial housing portion is illustrated in FIG. 4. With regard to the blood pump 1, FIG. 4 thus shows the housing 2 and the rotor 20 arranged in the interior 16. The cylinder 24 and the blading 28 and the ring 32 running externally around the blading can be seen. A gap of gap width r exists between the ring and the inner wall of the housing 2. This gap width defines whether or not a hydrodynamic bearing is formed between the outer surface of the ring 32 and the inner surface of the housing 32. In addition, the magnetised ring 14 is provided in the housing 2 and, in conjunction with the ring 32, provides an axial, passive mounting of the rotor in the housing 2. It can additionally be seen in FIG. 4 that the radius R of the rotor 20 is much greater than the width b of the ring 32.

The blading 28 in the present example comprises three spirals 50, 52 and 54, the height of which as measured in the radial direction is much smaller than the radius R of the rotor. The width B of an individual spiral is of such a size here that it is smaller than the distance between two adjacent spirals, for example the spirals 52 and 56. In FIG. 5 the rotor 20 is again illustrated in a three-dimensional perspective. The cylinder 24 and the ring 32 and the spiral 52, which along the length L of the rotor 20 turns helically around the outer surface of the cylinder 24 by at least 270°, preferably by at least 360°, can be seen. The rotor shown in FIG. 2 would be provided accordingly with a further ring at its downstream end.

Although in FIGS. 1, 2 and 3 the magnetised rings arranged in the axial housing portion were previously in each case complete rings, they could also be composed of individual segments. Here, completely closed rings are indeed preferred in some exemplary embodiments, however merely ring portions, i.e. angular segments of less than 360°, or 270°, 180° or 90°, could also be used.

A further embodiment of a blood pump is shown in FIG. 6. The blood pump 300 comprises a housing 302 with an inlet 304 and an outlet 306, which comprises a spiral chamber 308, as described for example in conjunction with FIG. 3. A mandrel 312 extends along the axis 310 from the outlet-side end of the pump and has a cylindrical portion 314, which extends between the upstream end 316 of the mandrel 312, shaped in the form of a segment of a sphere, and the spiral chamber 308. A stator 318 is disposed inside the mandrel and interacts with permanent magnets, which are arranged in the rotor 330, in order to set the rotor 330 in rotation.

The rotor 330 comprises a cylinder bushing 332, in which for example permanent magnets as described in US 2014/0171727 are arranged. Alternatively or additionally hereto, permanent magnets can also be used in the blading 334. The blading 334 can be formed for example as described in the previous examples. Alternatively, the rotor can also be formed in such a way that the width between the individual spirals is wider than a distance between two adjacent spirals. A first ring 336 and a second ring 338 arranged downstream of said first ring are disposed on the radially outer circumference of the blading 334. These rings interact with two rings 340 and 342, which are formed in the housing 302. The rings 340 and 342 each comprise two parts (a first part 344 and a second part 346), which are axially magnetised in opposite directions. This type of arrangement ensures that the rotor 330 is held on the mandrel. The cooperation of the rings 336 and 338 with the rings 340 and 342 respectively thus forms a passive axial mounting 350. The magnetic two-part embodiment of the rings 340 and 342 can also be used in pumps without a mandrel. Further components necessary for the pump are not shown in the illustration of FIG. 6.

FIG. 7 shows a cross-section through the arrangement of FIG. 6. In FIG. 7 the housing 302 and the mandrel 312 and the rotor 330 can be seen. A ring 340 is arranged running around the housing 302. The rotor comprises a cylinder bushing 302, a blading 334, and a first ring 336, which in conjunction with the ring 340 provides an axial bearing of the rotor 330 on the mandrel 312. A radial bearing between the mandrel and the rotor is formed via the gap between the outer surface of the mandrel 312 and the inner surface of the cylinder bushing 332. The gap width is of such a size that a hydrodynamic bearing is formed which stabilises the rotor radially. The stator 318, which drives the rotor, can also be seen in FIG. 6. Further exemplary embodiments of the pump shown here with axial bearing by means of a ring arranged on the outer edge of the blading can be found for example in the application having internal file reference 157EP 0367.

1. A blood pump comprising:
a housing with an inlet arranged upstream, an outlet arranged downstream; and
a rotatably mounted rotor having an axis and a blading, the rotor mounted in an axial direction, wherein at least one first ring is secured to the blading, runs radially externally around the blading, and is magnetised in the axial direction, and a second magnetised ring portion running externally around the first ring for forming an axial magnetic bearing is also arranged in a first axial housing portion, and the rotor comprises a cylinder bushing or a cylinder, wherein the blading is arranged on an outer surface of the cylinder bushing or the cylinder.

2. The blood pump of claim 1, wherein a second axial portion of the blading has a third ring portion and a second axial housing portion has a fourth ring portion corresponding to the third ring portion.

3. The blood pump of claim 1, wherein the second ring portion forms a closed ring.

4. The blood pump of claim 1, wherein the blading comprises a spiral.

5. The blood pump of claim 4, wherein the blading comprises a plurality of spirals and two adjacent spirals have a distance along an outer surface of the rotor that is a multiple of a width of the spirals.

6. The blood pump of claim 1, wherein the rotor is mounted in the axial direction exclusively by the axial magnetic bearing.

7. The blood pump of claim 1, wherein the axial magnetic bearing is formed in such a way that a downstream end of the blading is arranged fully upstream of the outlet.

8. The blood pump of claim 1, wherein the blading is arranged on the rotor in such a way that a downstream end of the rotor lies downstream of a downstream end of the blading.

9. The blood pump of claim 1, wherein a radial distance between an inner surface of the housing and an outer surface of the first ring is such that a hydrodynamic bearing is formed between the inner surface of the housing and the outer surface of the first ring.

10. The blood pump of claim 1, wherein a radial distance between an inner surface of the housing and an outer surface of the first ring is such that there is no hydrodynamic bearing formed between the inner surface of the housing and the outer surface of the first ring.

11. The blood pump of claim 1, wherein the outlet defines an outlet direction rotated (inclined) relative to the axis by an angle of more than 45°, preferably between 80° and 100°.

12. The blood pump of claim 1, wherein a mandrel, on which the rotor is rotatably mounted, is arranged between the inlet and the outlet.

13. The blood pump of claim 12, wherein a hydrodynamic bearing is formed between a surface of the mandrel and an inner surface of the rotor.

14. The blood pump of claim 12, wherein a stator driving the rotor is arranged in the mandrel and/or in the first housing portion and/or proximally of the rotor.

15. The blood pump of claim 12, wherein the mandrel and an interior defined by the housing are oriented coaxially with one another at least in part.

16. The blood pump of claim 1, wherein the first ring and/or the second ring portion comprise/comprises soft iron.

17. The blood pump of claim 1, wherein the blood pump is for assisting a heart.

18. The blood pump of claim 1, wherein the rotor is mounted magnetically in the axial direction.
